(19) United States
(12) Patent Application Publication
(10) Pub. No.: US 2007/0044179 A1
(43) Pub. Date: Feb. 22, 2007

(54) Insecticidal compositions and methods of using the same

(75) Inventors: C. Neal Stewart JR., Knoxville, TN (US); Brian M. Leckie, Knoxville, TN (US)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: University of Tennessee Research Foundation, Knoxville, TN (US)

(21) Appl. No.: 11/506,502

(22) Filed: Aug. 18, 2006

Related U.S. Application Data
(60) Provisional application No. 60/709,259, filed on Aug. 18, 2005.

(51) Int. Cl. A01H 1/00; C12N 15/87; A01N 63/00

(52) U.S. Cl. 800/279; 424/93.461

(57) ABSTRACT

The subject application concerns novel materials and methods for controlling non-mammalian pests. In a certain embodiments, the subject materials and methods for the control of coleopteran or lepidopteran pests are provided. The subject application also provides pesticidal proteins and compositions comprising pesticidal proteins that are derived from Beauvaria bassiana. These proteins have molecular weights that are greater than about (or greater than) 5000 daltons. In a preferred embodiment, the subject invention concerns plants cells, plant parts or plants to which the pesticidal proteins, or compositions thereof, have been applied.

### CROSS-REFERENCE TO RELATED APPLICATION

This application claims the benefit of U.S. Provisional Application Ser. No. 60/709,259, filed Aug. 18, 2005, the disclosure of which is hereby incorporated by reference in its entirety.

### BACKGROUND OF THE INVENTION

Every important crop and horticultural plant experiences damage as a result of herbivory from insects. Phytophagous insects consume 30% of the world's total crop yield each year. These amounts correspond to well over 100 billion dollars (US) in annual losses (Basra and Basra, 1997). Genes encoding orally toxic proteins have been cloned from the insect pathogen Bacillus thuringiensis (Bt) and transformed into crops to confer insect resistance. Bt transgenic plants are engineered to produce a variety of proteinacious delta endotoxins (Peferoen 1997).

In 2003, 67.7 million hectares of transgenic crops were grown worldwide. Bt crops represented 18 percent or 12.2 million hectares, making Bt insect resistance the second most utilized trait in GM crops (James 2003). While there have been no reports in natural populations, lab-reared insects under heavy selection have developed resistance (Tabashnik 1994; Tabashnik et al. 1996; Perez and Shelton 1997; Tabashnik et al. 1997). These studies and the global reliance on Bt crops for insect control has spurred concerns that insects may soon develop resistance in field populations. These concerns have prompted companies toward increasing gene discovery activities due to the need for a greater battery of insecticidal genes and proteins (Stewart 1999).

Other orally toxic proteins have been utilized in transgenic plants. These toxins represent a wide variety of protein classes. Proteinase inhibitors interfere with digestive enzymes in the insect gut and have been shown to be effective in reducing insect herbivory in transgenic crops (Hoffman et al. 1992; Hilder et al. 1987; Sane et al. 1997; Xu et al. 1996). Tobacco transformed with a cholesterol-oxidase gene was toxic to the boll weevil (Anthonomus grandis) by damaging the midgut epithelium (Corbin et al. 2001). A gene from the bacteria Photorhabdus luminescens encoding the TcdA protein, when transformed into Arabidopsis thaliana, exhibited a high level of toxicity to the tobacco hornworm (Manduca sexta) (Liu et al. 2003). Lectins, which have sugar binding properties, have been transformed into oilseed rape and potato and have exhibited deleterious effects on pollen beetle (Meligethes aeneus) (Melander et al. 2003) and peach potato aphids (Myszus persicae) (Gatehouse et al. 1996), respectively. Insect chitinases (Ding et al. 1998) and α-amylase inhibitors (Schroeder et al. 1995) have also been utilized as candidates to reduce insect damage and have shown some success.

Beauveria bassiana (Balsamo) Vuillemin is a ubiquitous soil-inhabiting entomopathogenic fungus in the phylum Deuteromycota. A variety of insects, at all stages of development are susceptible hosts of B. bassiana (McCoy et al. 1985). Due to its wide host range of almost 500 susceptible species of insects (Vilcinskas and Gotz 1999), B. bassiana has been tested as a microbial control agent against most of the economically important insect pests. Pests that have been successfully controlled by B. bassiana include: the lesser stalk borer, Elasmopalpus lignosellus (McDowell et al. 1990), European corn borer, Ostrinia nubilalis (Bing and Lewis 1991; Feng et al. 1988), hop aphid, Phorodon humuli (Dorschner et al. 1991), greenhouse whitefly, Trialeurodes vaporariorum (Poprawski et al. 2000), and Colorado potato beetle, Leptinotarsa decemlineata (Jaros-Su et al. 1999). The efficacy of B. bassiana as a biological control agent against these insects demonstrates that the fungi's natural infection cycle, which includes the production of toxic compounds, is sufficient to cause significant mortality.

The infection cycle of B. bassiana in an insect begins with the contact of a conidium with the cuticle of a susceptible host. The conidium germinates and the fungus produces an array of enzymes that help degrade the outer integument. These enzymes include proteases, chitinases, and lipases. The fungus produces a germ tube that grows through the integument and toward the hemocoel. Once the hemocoel is entered, blastospore formation and toxin production begin (Boucias and Pendland 1988). As the fungus proliferates, the host dies and becomes mummified by hyphal growth that will later extrude from the cadaver through intersegmental membranes (Pekrul and Grula 1979). Death usually occurs in three to seven days and is attributed to nutrient deficiency, water loss, or the action of toxins (Boucias and Pendland 1988).

Research on B. bassiana has demonstrated that this fungus may be orally toxic when ingested by lepidopterans. The first study to shed light on the subject observed that B. bassiana was able to grow endophytically in corn (Poaceae) (Lewis and Bing 1991) and confer resistance to insect herbivory. After foliar application to corn plants at the V8 stage, B. bassiana was recovered from the pith of plants. Percentage of plants with recovered B. bassiana was negatively correlated (r=−0.376) with insect damage per plant. Over the two-year study, plants treated with B. bassiana exhibited suppression of tunneling by larval European corn borer (O. nubilalis) ranging from 37.0% to 50.6% (Lewis and Bing 1991). In a later study, granular formulations of B. bassiana conidia, applied to the foliage of corn at the whorl-stage, grew into and colonized up to 98.3% of plants (Bing and Lewis 1991). Once established in the plant, the fungus again decreased tunneling of O. nubilalis. Endophytic colonization of corn by B. bassiana showed no yield reduction or adverse effects on plants (Lewis et al. 1996).

Further investigation of the effects of ingested B. bassiana on insect pests involved corn earworm larvae (Helicoverpa zea) fed a synthetic diet containing dried mycelia of to B. bassiana isolates (3-00 and 11-98) (Leckie 2002). Delayed development and high mortality were observed in larvae fed the highest rates (1 and 5% w/v) of fungal diet. Weights of surviving larvae and pupae were also lower for larvae fed the higher concentrations of mycelia. After 10 days, larval mortality was 100% for the 5% mycelia diet treatment of one isolate (11-98), which was significantly greater than control diets and diets containing isolate 3-00. Some insects that died were observed to be stuck to the plastic cups by a translucent fluid which emanated from the anus of the insect. These observations may be similar to those noted by Ahmad et al. (1985), where house crickets, Acheta domesticus (Linnaeus), suffered from complete failure of the alimentary process due to feeding on perennial ryegrass infected with Neotyphodium loliae. These deleterious effects were attributed to the toxic compounds in the mycelium. Differences in the effects of different isolates were attributed to the relative amounts of toxins produced by each isolate. Variations in production of toxic metabolites have been documented for a variety of entomopathogenic fungi and are not unusual (Strasser et al. 2000).

The isolation of high molecular weight compounds produced by B. bassiana has revealed several toxic proteins. Two proteases were shown to be toxic when injected into Galleria mellonella (Kucera and Samainakova 1968). Another protein with a toxic effect when injected into Galleria mellonella, Bclp, was isolated from B. bassiana and was shown to induce cuticular melanization (Fuguet et al. 2004; Fuguet and Vey 2004). Bassiacridin, a protein showing similarity to a yeast chitin binding protein, was toxic at low dosages when injected into Locusta migratoria (Quesada-Moraga and Vey 2004). Although toxic when injected into the hemoceol, none of these proteins were evaluated for oral toxicity.

While only a few high molecular weight toxic compounds have been isolated from B. bassiana, a variety of low molecular weight toxic compounds have been reported. These include beauvericin, bassianolide, and the red pigmented toxin oosporein. Cyclosporin is also produced as a secondary metabolite and is a known immunosuppressant produced by other fungi (Boucias and Pendland 1998). Beauvericin, when injected into adult blowflies, Calliphora erythrocephala(Meig.), resulted in 15% mortality by day 2. When injected into larval yellow fever mosquitoes, Aedes aegypti, mortality reached 39% at 48 hours (Grove and Pople 1980). Suspensions of beauvericin added to water containing larval northern house mosquitoes, Culex pipiens autogenicus, killed 44% of the larvae by 48 hours (Zizka and Weiser 1993). Beauvericin, when applied to leaf disks and fed to Colorado potato beetles, had an LC₅₀ of 633 ppm and an LC₉₀ of 1196 ppm (Gupta et al. 1991). Conversely, beauvericin was shown to have no oral toxicity to silkworms at levels as high as 1000 ppm (Kanaoka et al. 1978). In this same study, bassianolide was also administered orally to silkworms and was lethal at 8 ppm. Fermentation broth obtained from the production of B. bassiana and containing the red pigment oosporein caused 49.8% mortality in mealy bugs feeding on topically applied leaves (Eyal et al. 1994).

### BRIEF SUMMARY OF THE INVENTION

The subject application concerns novel materials and methods for controlling non-mammalian or insect pests. In a certain embodiments, one invention provides materials and methods for the control of coleopteran or lepidopteran pests are provided. The subject application also provides pesticidal proteins and compositions comprising pesticidal proteins that are derived from Beauvaria bassiana. These proteins have molecular weights that are greater than about (or greater than) 5000 daltons. In a preferred embodiment, the subject invention concerns plants cells, plant parts or plants to which the pesticidal proteins, or compositions thereof, have been applied.

### BRIEF DESCRIPTION OF THE FIGURES

FIG. 1 shows the percent mortality of neonate diamondback moth larvae fed synthetic diets supplemented with Bt canola proteins, Beauveria bassiana proteins, or buffer added at 0.1, 1 or 10% total volume of diet. Bars represent standard error. Letters above bars represent significance levels of 0.05 under ANOVA and mean separation using LSD.

FIG. 2 demonstrates that insects feeding on diets containing six and twenty-eight day samples suffered significantly greater mortality (72 and 80%) than those feeding on protease-digested samples (45 and 42%). Insects feeding on buffer control diets suffered statistically similar mortality to those fed protease-treated buffer diet. This indicates that the addition of pronase to samples had no significant effect on mortality. These results clearly demonstrate that the protein components of the extracts were responsible for a significant increase in insect mortality, and that the toxic protein components are present in both six and twenty-eight day fungal growth.

FIG. 3 illustrates a bioassay that is conducted in three tiers. In tier 1 the individual cDNAs can be pooled into batches/lots containing 100-200 individual cDNAs from B. bassisana and can produce approximately 150 batches/lots. A batch/lot having high oral toxicity is then subdivided into batches/lots containing 10-20 different cDNAs each for the tier 2 feeding study (approximately 15 batches/lots). Any batch/lot exhibiting oral toxicity is further subdivided for the tier 3 feeding study into batches/lots containing only one cDNA each. Batches/lots denoted with an asterisk (*) indicate high oral toxicity.

### DETAILED DISCLOSURE OF THE INVENTION

One aspect of the subject invention concerns novel protein extracts obtained from B. bassiana, preferably B. bassiana, strain 11-98. These protein extracts have been processed and comprise proteins and other components having molecular weights of at least about (or at least) 5000 daltons.

The protein extracts described herein can be formulated into compositions. Compositions comprising the disclosed protein extracts can be in solid form (e.g., granule, powder, or dust), liquid form, of formulated for dispersion as an aerosol. Compositions comprising the protein extracts of the subject invention can further comprise one or more (or at least one) additional pesticidal proteins or compounds (e.g., Bacillus thuringiensis (Bt) toxins or portions thereof).

Compositions comprising the protein extract (and optionally one or more additional pesticidal protein) of the subject invention can also contain an attractant and spores or crystals of Bt isolates. Alternatively, recombinant microbes comprising the genes obtainable from the Bt isolates known to be insecticidal can be used in the formulation of a composition according to the subject invention. Formulated compositions can also be applied as a seed-coating, root treatment or total plant treatment at later stages of the crop cycle. Plant and soil treatments of using the compositions of the subject invention may be employed as wettable powders, granules or dusts, by mixing with various inert materials, such as inorganic minerals (phyllosilicates, carbonates, sulfates, phosphates, and the like) or botanical materials (powdered corncobs, rice hulls, walnut shells, and the like). The formulations may include spreader-sticker adjuvants, stabilizing agents, other pesticidal additives, or surfactants. Liquid formulations may be aqueous-based or non-aqueous and employed as foams, gels, suspensions, emulsifiable concentrates, or the like. The ingredients may include rheological agents, surfactants, emulsifiers, dispersants, or polymers.

As would be appreciated by a person skilled in the art, the pesticidal concentration of the protein extract described herein will vary widely depending upon the nature of the particular formulation, particularly whether it is a concentrate or to be used directly. The pesticidal protein extract will be present in at least 1% by weight and may be 100% by weight of a given composition formulation. Certain embodiments provide that the protein extract of the subject invention constitute at least about (or at least) 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% of a given composition formulation. The dry formulations will have from about 1-95% by weight of the pesticide while the liquid formulations will generally be from about 1-60% by weight of the solids in the liquid phase. The formulations can be applied to the environment of the pest, e.g., soil and foliage, by spraying, dusting, sprinkling, or the like.

The subject invention also provides methods of treating plants, plant cells or plant parts comprising the application of the protein extract of the subject invention, or compositions thereof, to at least one plant, plant cell or plant part (e.g., roots, stems, leaves, etc.). As would be apparent to the skilled artisan, the protein extract or compositions of the subject invention can also be applied to entire fields of plants or plant parts (e.g., field crops such as corn, maize, canola, soybean, tobacco, or cotton).

The subject invention also provides methods of controlling a non-mammalian or insect pest comprising the application of the protein extract (or compositions thereof) of the subject invention to at least one plant, plant cell or plant part. In this aspect of the invention, the protein extract or composition thereof is applied to at least one plant, plant part or plant cell in an amount sufficient to control the non-mammalian plant or insect pest population, reduce the number of non-mammalian plant or insect pests or eradicate the non-mammalian plant pest. As discussed supra, the protein extract (or compositions thereof) provided by the subject invention can be applied to entire fields of plants or plant parts. In certain aspects of the subject invention, the non-mammalian plant or insect pest is a coleopteran or lepidopteran pest. One non-limiting example of a lepidopteran pest is the diamondback moth, Plutella xylostella.

Host cells to be used in screening assays as set forth herein may be chosen from eukaryotic or prokaryotic systems, such as for example bacterial cells, (Gram negative or Gram positive), yeast cells (for example, Saccharomyces cereviseae or Pichia pastoris), animal cells (such as Chinese hamster ovary (CHO) cells), plant cells, and/or insect cells using baculovirus vectors. In some embodiments, the host cells for expression of the polypeptides include, and are not limited to, those taught in U.S. Pat. Nos. 6,319,691, 6,277,375, 5,643,570, or 5,565,335, each of which is incorporated by reference in its entirety, including all references cited within each respective patent.

Furthermore, a host cell strain may be chosen which modulates the expression of the inserted sequences, or modifies and processes the gene product in the specific fashion desired. Expression from certain known promoters can be elevated in the presence of certain inducers; thus, expression of the genetically engineered polypeptide may be controlled. Furthermore, different host cells have characteristic and specific mechanisms for the translational and post-translational processing and modification (e.g., glycosylation, phosphorylation) of proteins. Appropriate cell lines or host systems can be chosen to ensure the desired modification and processing of the foreign protein expressed. For example, expression in a bacterial system can be used to produce an unglycosylated core protein product. Expression in yeast will produce a glycosylated product. Expression in mammalian cells can be used to ensure “native” glycosylation of a heterologous protein. Furthermore, different vector/host expression systems may effect processing reactions to different extents.

B. bassiana strain 11-98 has been deposited with the Agricultural Research Service Culture Collection (National Center for Agricultural Utilization Research, 1815 N. University Street, Peoria, Ill. 61604 USA) as NRRL-30872 under conditions that assure that access to the cultures will be available during the pendency of this patent application to one determined by the Commissioner of Patents and Trademarks to be entitled thereto under 37 C.F.R. 1.14 and 35 U.S.C. 122. The deposits are available as required by foreign patent laws in countries wherein counterparts of the subject application, or its progeny, are filed. However, it should be understood that the availability of a deposit does not constitute a license to practice the subject invention in derogation of patent rights granted by governmental action.

Further, the subject culture deposits will be stored and made available to the public in accord with the provisions of the Budapest Treaty for the Deposit of Microorganisms, i.e., they will be stored with all the care necessary to keep them viable and uncontaminated for a period of at least five years after the most recent request for the furnishing of a sample of a deposit, and in any case, for a period of at least 30 (thirty) years after the date of deposit or for the enforceable life of any patent which may issue disclosing the cultures. The depositor acknowledges the duty to replace the deposit(s) should the depository be unable to furnish a sample when requested, due to the condition of the deposit(s). All restrictions on the availability to the public of the subject culture deposits will be irrevocably removed upon the granting of a patent disclosing them.

Thus, this application provides the following non-limiting embodiments:

1. A composition of matter comprising a plant or plant part to which a composition comprising an extract of Beauvaria bassiana 11-98 (NRRL-30872) has been applied, said extract comprising insecticidal protein components having molecular weights of greater than 5000 daltons and said extract being insecticidal;

2. The composition of matter according to embodiment 1, wherein one or more additional pesticidal protein or pesticidal compound has been applied to said plant or plant part;

3. The composition of matter according to embodiment 2, wherein said one or more additional pesticidal protein or pesticidal compound is one or more insecticidal Bacillus thuringiensis (Bt) toxin or insecticidal portions thereof;

4. The composition of matter according to embodiments 1, 2 or 3, wherein said composition further comprises an attractant and spores or crystals of Bacillus thuringiensis isolates;

5. A composition comprising an extract of Beauvaria bassiana, said extract comprising insecticidal protein components having molecular weights of greater than 5000 daltons and said extract being insecticidal when ingested by an insect;

6. The composition according to embodiment 5, wherein said composition further comprises one or more additional pesticidal proteins or compounds;

7. The composition according to embodiment 6, wherein said composition further comprises one or more insecticidal Bacillus thuringiensis (Bt) toxin or insecticidal portions thereof;

8. The composition according to embodiments 5, 6 or 7, wherein said composition further comprises an attractant and spores or crystals of Bacillus thuringiensis isolates;

9. The composition according to embodiments 5, 6, 7, or 8, wherein said extract is an extract of Beauvaria bassiana 11-98 (NRRL-30872);

10. A method of treating a plant, plant cell, or plant part comprising the application of a composition comprising an extract of Beauvaria bassiana, said extract comprising insecticidal protein components having molecular weights of greater than 5000 daltons and said extract being insecticidal to a plant, plant cell, or plant part;

11. The method according to embodiment 10, wherein said composition further comprises one or more additional pesticidal proteins or compounds;

12. The method according to embodiments 10 or 11, wherein said composition further comprises one or more insecticidal Bacillus thuringiensis (Bt) toxin or insecticidal portions thereof;

13. The method according to embodiments 10, 11 or 12, wherein said composition further comprises an attractant and spores or crystals of Bacillus thuringiensis isolates;

14. The method according to embodiments 10, 11, 12, or 13, wherein said extract is an extract of Beauvaria bassiana 11-98 (NRRL-30872);

15. A method of controlling an insect pest comprising the application of a composition comprising an extract of Beauvaria bassiana, said extract comprising insecticidal protein components having molecular weights of greater than 5000 daltons and said extract being insecticidal;

16. The method according to embodiments 15, wherein said composition further comprises one or more additional pesticidal proteins or compounds;

17. The method according to embodiments 15 or 16, wherein said composition further comprises one or more insecticidal Bacillus thuringiensis (Bt) toxin or insecticidal portions thereof;

18. The method according to embodiments 15, 16 or 17, wherein said composition further comprises an attractant and spores or crystals of Bacillus thuringiensis isolates;

19. The method according to embodiments 15, 16, 17, or 18, wherein said extract is an extract of Beauvaria bassiana 11-98 (NRRL-30872);

20. The method according to embodiments 15, 16, 17, 18 or 19, wherein said insect pest is coleopteran or lepidopteran;

21. A method of screening for an insecticidal protein comprising:

(a) transforming a host cell with one or more nucleic acid encoding B. bassiana proteins;

(b) culturing said host cell under conditions that cause the overexpression of said B. bassiana proteins;

(c) purifying B. bassiana proteins from said host cell or culture media;

(d) screening for at least one insecticidal protein comprising feeding an insect a diet comprising into which said at least one B. bassiana protein as been incorporated to form a protein modified insect diet and comparing the mortality of said protein modified insect diet on tested insects against a one or more of the following controls:
(i) a control diet into which no insecticidal proteins have been incorporated;
(ii) a control diet into which protein extracts from non-transformed host cells have been incorporated; or
(iii) a control diet into which protein extracts from host cells with a vector containing no nucleic acid inserts encoding B. bassiana proteins have been incorporated;
   wherein an insecticidal B. bassiana protein is identified when increased in mortality is observed in insects tested with said protein modified insect diet as compared to the mortality observed in insects fed one or more of the control diets;
   22. The method according to embodiment 21, wherein said one or more controls is selected from: (i); (ii); (iii); (i) and (ii); (i) and (iii); (ii) and (iii); or (i), (ii) and (iii);
   23. The method according to embodiment 21 or 22, wherein said host cell is a yeast cell;
   24. The method according to embodiment 21, 22 or 23 wherein said modified insect diet comprises a diet into which a pool of B. bassiana proteins is incorporated;
   25. The method according to embodiment 21, 22, 23 or 24, wherein said pool of B. bassiana proteins comprises 100 to 200 recombinant proteins;
   26. The method according to embodiment 21 22, 23, 24 or 25, wherein said insects are coleopteran or lepidopteran; or
   27. The method according to embodiments 21, 22, 23, 24 or 25, wherein said insects are diamondback moths (Plutella xylostella), fall armyworms (Spodoptera frugiperda), corn earworms (Helicoverpa zea), southern corn rootworms (Diabrotica undecimpunctata), Colorado potato beetle (Leptinotarsa decemlineata), Asian tiger mosquitoes (Aedes albopictus), or green peach aphids (Myzus persicae).

The terms “comprising”, “consisting of” and “consisting essentially of” are defined according to their standard meaning. The terms may be substituted for one another throughout the instant application in order to attach the specific meaning associated with each term.

Following are examples which illustrate procedures for practicing the invention. These examples should not be construed as limiting. All percentages are by weight and all solvent mixture proportions are by volume unless otherwise noted.

### EXAMPLE 1

In order to confirm that the observed toxicity of isolate 11-98 is protein based, a bioassay involving the addition of isolated proteins into synthetic insect diet was performed. This isolation procedure removes the low molecular weight metabolites to more accurately evaluate the proteins. B. bassiana isolate 11-98 conidia were used to inoculate 1 L of three media types SDY (peptone, dextrose, and yeast extract), YG (yeast extract and dextrose), and PG (peptone and dextrose). Fungal cultures were allowed to grow for one month. Mycelia were then collected by centrifugation at 5000 rpm for 5 minutes. Proteins were extracted from mycelial tissue by standard HEPES buffer protein extraction procedure. All protein extracts were dialyzed for 24 hours using a 3500 Molecular Weight Cutoff Slidealyzer cassette (Pierce. Biotechnology) to remove any secondary metabolites and then concentrated in a VIVASPIN spin concentrator (Molecular Weight Cutoff 5000 Vivascience, Cat# VS2011). Extracts were also quantified by Bradford assay and standardized to 10 mg/ml protein.

Proteins were also extracted from two positive controls, wild type canola var. westar, and Saccharomyces cerevisiae. These proteins and two other positive controls, water and buffer were processed in the same manner. Processed protein from a negative control, GT₂ transgenic Bt canola, was also included. Protein extracts were then added to synthetic diamondback moth diet (Bio-serve) at rates of 0.1, 1, and 10% total volume. Water and buffer diets were made to control for the standardized quantities of proteinacious liquids added to the other diets. Two 1-cm³ pieces of a single diet type were added to a plastic cup containing a moist filter paper. Ten neonate diamondback moths were added to each container. Three replicates of each diet type were included in the bioassay. Cups were kept at room temperature for eleven days. Surviving insects were then counted. ANOVA were used to evaluate differences in insect survivorship and Fisher's PLSD (P=0.05) were used to determine statistical differences between diet types and rates.

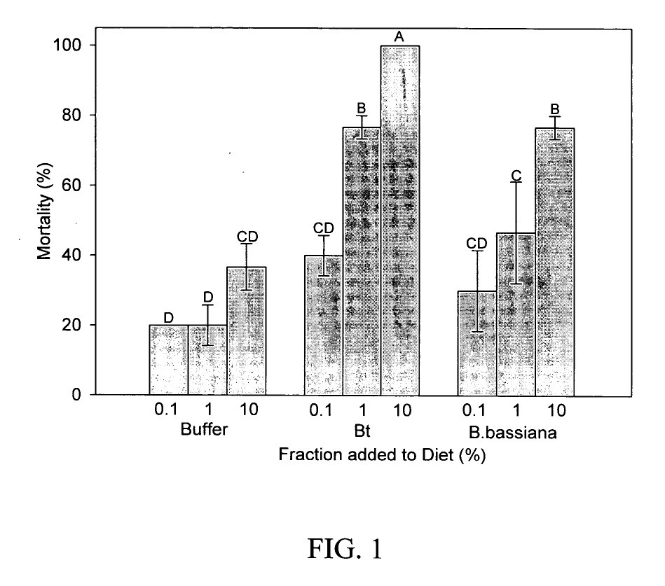

Insects feeding on the high rate of SDY diet suffered a significantly greater mortality (76.6%) than those insects feeding on all positive control diets (water, buffer, westar, and yeast) and similar mortality to those insects fed the intermediate rate of Bt canola proteins (FIG. 1). Insects fed fungal proteins cultured on media types other than SDY resulted in lower mortality. As expected, only insects fed the GT₂ Bt canola diet at the highest rate suffered greater mortality than the insects fed the high rate of SDY diet, this is due to the high levels of Bt Cry1ac produced in the GT₂ transgenic line. Some insects that died were attached to the plastic cups by a translucent fluid, which emanated from the anus of the insect. This observation is consistent with previous observations (Leckie 2002) and may indicate that this toxic mode of action results from ingestion of the fungal proteins.

### EXAMPLE 2

To further demonstrate that the toxicity is protein based, a bioassay containing extracts subjected to protease digestion was performed. Beauveria bassiana 11-98 was grown for durations of six and twenty-eight days and then harvested. Proteins were isolated in the previously described manner and standardized to 10 mg/ml. One milliliter of extraction buffer, six day, and twenty-eight day samples were digested with pronase (Roche Applied Science, Cat# 10 165 921 001), according to the manufacturer's directions. The protease was then heat inactivated. Digested extracts and undigested extracts were added to synthetic diamondback moth diet at a rate of 0.5 mg/ml. The bioassay and analysis was performed as previously described, except that each diet type had 10 replicates for a total of 100 insects per diet type.

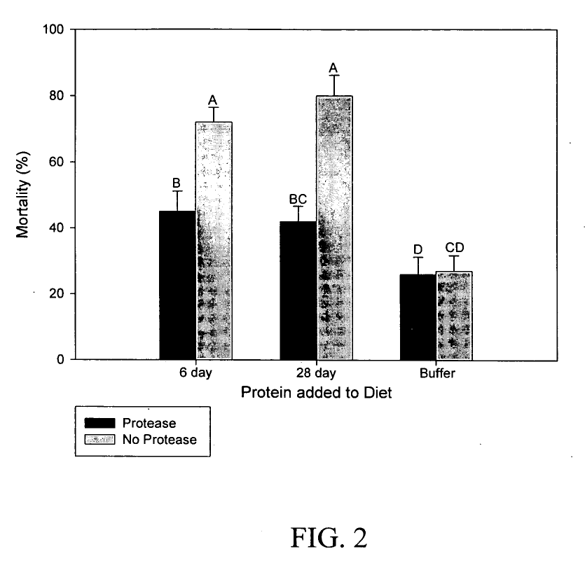

Insects feeding on diets containing six and twenty-eight day samples suffered significantly greater mortality (72 and 80%) than those feeding on protease-digested samples (45 and 42%) (FIG. 2). Insects feeding on buffer control diets suffered statistically similar mortality to those fed protease-treated buffer diet. This indicates that the addition of pronase to samples had no significant effect on mortality. These results clearly demonstrate that the protein components of the extracts were responsible for a significant increase in insect mortality, and that the toxic protein components are present in both six and twenty-eight day fungal growth.

### EXAMPLE 3

A cDNA library expressed in yeast is used for a screen using insect bioassays. The fungal gene expressing a toxin is isolated by sequential pooling of random cDNAs as described below.

Fungi

Beauveria bassiana isolate 11-98 conidia is used to inoculate 1 L of SDY media. The culture is allowed to grow for 10 days. mRNA is be extracted from harvested mycelium.

mRNA Extraction

The fresh mycelium is harvested, snap frozen in liquid nitrogen, and ground in a mortar and pestle. Total RNA is extracted using Trizol reagent (Invitrogen, Carlsbad, Calif.) according to the manufacturers protocol. mRNA is isolated by binding total RNA to Dynal Oligo-dT₂₅ magnetic beads (Dynal, Oslo, Norway) according to the manufacturer's instructions. The isolated mRNA is quantified and used in the cDNA synthesis reactions.

cDNA Library Construction

cDNAs are synthesized from mRNA using a CloneMiner cDNA synthesis kit (Invitrogen, Carlsbad, Calif.) and placed in the included pDONR222 vector by manufacturer's instructions. Escherichia coli ElectroMax DH10B T-1 cells (Invitrogen, Carlsbad, Calif.) are transformed by electroporation in a 2 mm gap cuvette with a BioRad Gene Pulser set at 2.5 kV, 25 μFD and 200 ohms (BioRad, Richmond, Calif.) with the library. The library of cDNAs is then extracted by Qiagen plasmid Maxi Kit (Qiagen, Valencia, Calif.). The library is transferred in bulk into the pYes-DEST52 yeast expression vector (Invitrogen, Carlsbad, Calif.) by a recombination reaction.

Subclone cDNAs into Yeast Expression Vectors in Lots and Induce Recombinant Expression.

Yeast (Saccharomyces cerevisiae) strain INVSc1 (MATα his3Δ1 leu2 trp1-289 ura3-52) is used (Invitrogen, Carlsbad, Calif., USA). A yeast expression vector, pYES-DEST52 (Invitrogen, Carlsbad, Calif.), which contains a URA3 gene for selection of transformants in yeast host strains with the ura3 genotype, is transformed into INVSc1 by the high-efficiency direct yeast transformation method (Adams et. al., 1998; Gietz and Schiestl 1995). This method utilizes lithium acetate to simply and efficiently generates transformed yeast cells in a 24 h period. Transformed cells are selected on synthetic complete (SC) medium lacking uracil for the selection of URA3 transformants (SC-uracil media, Sigma, St. Louis, Mo., USA).

The B. bassiana cDNA library in pYES-DEST52 is transformed into the yeast strain INVSc1 as above. The original transformation includes all cDNAs. The cDNA/pYES2 library is grown in 125 ml cultures that contain a population of individual fungal cDNA fragments, and replicate populations are established to ensure the inclusion a large number of the cDNA sequences. Yeast is also be plated for the propagation of individual colonies containing a single cDNA. These colonies are picked and pooled into lots of 100 to 1000 colonies per culture.

Expression of cDNA sequences fused to the GAL1 promoter will be upregulated in the presence of galactose and repressed by glucose, and therefore, the yeast transformants are subjected to SC-uracil media containing 2% galactose or 2% glucose, respectively. The 2% glucose medium serves as an important negative control to ensure that colonies that form on the 2% galactose medium are the result of the expression of the plant cDNA. The yeast cDNA populations are grown to exponential phase cells in 125 ml cultures of liquid SC-uracil media in 500 mL-beveled flasks and proteins are extracted. Recombinant proteins are expressed at a rate up to 100 μg/ml and are between 0.5 and 0.9% of the yeasts total protein (Invitrogen technical support).

Pool Recombinant Fungal Proteins and Incorporate them into Diet to Perform Subsequent Bioassays Against Economically Important Insects.

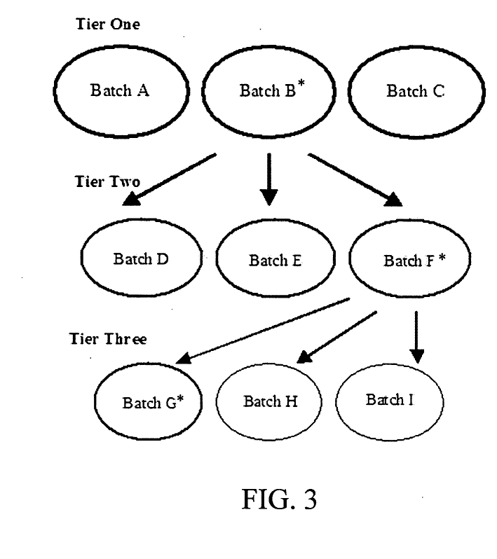

The lots of 100 to 200 different recombinant proteins are extracted from yeast colonies to use as incorporated components in insect diet. Target insect mortality is the primary variable to determine which lots of clones contain lethal proteins. These lots are further divided into increasingly smaller lots to determine which cDNA clone(s) is/are responsible for encoding toxic protein(s) (see FIG. 3).

Insects

Diamondback moths (DBM) (Plutella xylostella) are used in the initial stages of screening. DBM eggs are purchased from reliable sources Agripest (Zebulon, N.C.) or French Agricultural Research, Inc. (Lamberton, Minn.) and reared to second larval instar stage before being transferred to Petri dishes for bioassays. In subsequent experiments, fall armyworm (Spodoptera frugiperda), corn earworm (Helicoverpa zea), southern corn rootworm (Diabrotica undecimpunctata), and Colorado potato beetle (Leptinotarsa decemlineata) are used to evaluate candidate proteins by similar assays. Other economically important insects such as Asian tiger mosquito (Aedes albopictus) and green peach aphid (Myzus persicae) are also targets for other bioassays.

Protein

Yeast cultures of Saccharomyces cerevisiae strain INVSc1 transformed with fungal cDNA are grown overnight in 1 L flasks containing SC-ura+gal to an OD₆₀₀ of 2. Cells are then collected by centrifugation at 3000×G for 5 min at 4° C. Proteins are extracted from mycelial tissue by standard Hepes buffer protein extraction procedure. All protein extracts are dialyzed for 24 hours using a 3500 dalton MWCO Slidealyzer cassette (Pierce Biotechnology, Rockford, Ill.) to remove any secondary metabolites and then concentrated in a VIVASPIN spin concentrator MWCO 5000 dalton (Vivascience, Cat# VS2011). Extracts are also quantified by Bradford assay and standardized to 10 mg/ml protein. Proteins are extracted from non-transformed Saccharomyces cerevisiae and Saccharomyces cerevisiae transformed with the Bt Cry1Ac gene, as controls for comparison. Protein is immediately quantified and added to diets for testing.

Diet

Artificial diamondback moth (#F9441B) diet mix is obtained from Bio-Serv Inc. (Frenchtown, N.J.). Aliquots of protein solutions in multiple volumes are incorporated into the synthetic diet. The diet is made by the manufacturer's instructions and protein solutions added while the diet is setting. Protein solutions will be added to the diet at concentrations of 1, 0.1, and 0.01 mg/ml. Diets with additives will be mixed vigorously, and stored in a refrigerator (4° C.) until used. Additional control diets will also be prepared containing extraction buffer, water, Bt toxins or yeast proteins.

Bioassays

The cDNAs from yeast populations will be evaluated in insect feeding tests containing protein extract supplemented artificial diet. Three rates (1, 0.1, and 0.01 mg/ml) of proteins will be tested. Neonate DBM are exposed to the protein extract supplemented artificial diet and the 3 control diets. A moist #2 Whatman filter paper (Maidstone, England) is placed in the bottom of a 100×15 mM petri dish (VWR International, So. Plainfield, N.J.) and a 2 cm³ piece of diet added to each plate. Ten larval insects are placed into each plate. Each treatment has three replicates; each replicate consists of ten insects per plate. Plates are placed in an incubator at 12 h/12 h photoperiod and held at 24° C. Additional diet is added to the plates when needed, to provide an excess of diet and to maintain freshness. Larvae are observed at four, eight, and twelve days and mortality is recorded. Developmental abnormalities are recorded and further explored. ANOVA is used to evaluate differences in insect mortality and Fisher's PLSD is used to determine statistical differences between diet types and rates. Mortality in a container results in additional rounds of screening, now with groups of 10 clones, and so forth until clone(s) encoding toxic protein(s) are delineated. The putative clones are sequenced and their functions are compared to other cloned sequences using BLAST. Clones that confer resistance to DBM are tested on a battery of other economic insects as above in incorporated synthetic diets or as leaf painting assays as indicators for efficacy of control.

### REFERENCES

Adams, A., Gottschling, D. E., Kaiser, C. A., and Stearns, T. (1998) “Methods in yeast genetics. A cold spring harbor laboratory course manual” Cold Spring Harbor Laboratory Press, Cold Spring Harbor, N.Y.
Basra, A. Basra, R. (1997) Mechanisms of Environmental Stress Resistance in Plants. Harwood Academic Publishers, Amsterdam. pp. 373-403.
Bing, L. A., and Lewis, L. C. (1991) “Suppression of Ostrinia nubilalis (Hubner) (Lepidoptera: Pyralidae) by endophytic Beauveria bassiana (Balsamo) Vuillemin” Environmental Entomology 20:1207-1211.
Boucias, D. G., Farmerie, W. G., and Pendland, J. C. (1998) “Cloning and sequencing of cDNA of the insecticidal toxin Hirsutellin A” Journal of Invertebrate Pathology 72:258-261.
Boucias, D. G., and Pendland, J. C. (1998) Principles of Insect Pathology, Kluwer Academic Publishers, Boston.
Corbin, D. R., Grebenok, R. J., Ohnmeiss, T. E., Greenplate, J. T., and Purcell, J. P. (2001) “Expression and chloroplast targeting of cholesterol oxidase in transgenic tobacco plants” Plant Physiology 126:1116-1128.
Dorschner, K. W., M. G. Feng, and Baird, C. B. (1991) “Virulence of an aphid-derived isolate of Beauveria bassiana (Fungi: Hyphomycetes) to the Hop aphid Phorodon humuli (Homoptera: Aphididae)” Environmental Entomology 20:690-693.
Ding, X. F., Gopalakrishnan, B., Johnson, L. B., White, F. F., Wang, X. R., Morgan, T. D., Kramer, K. J., and Muthukrishnan, S. (1998) “Insect resistance of transgenic tobacco expressing an insect chitinase gene” Transgenic Research 7:77-84.
Eyal, J., Mabud, MD. A., Fischbein, K. L., Walter, J. F., Osborne, L. S., and Landa, Z. (1994) “Assessment of Beauveria bassiana Nov. EO-1 strain, which produces a red pigment for microbial control” Appl. Biochem. Biotechnol. 44:65-80.
Feng, Z., Carruthers, R., Larkin, T., and Roberts, D. (1988) “A phenology model and field evaluation of Beauveria bassiana (Bals.) Vuillemin (Deuteromycota: Hyphomycetes) mycosis of the European corn borer, Ostrinia nubilalis (Hbn.) (Lepidoptera: Pyralidae)” Canadian Entomologist 120:133-144.
Fuguet, R., Theraud, M., and Vey, A. (2004) “Production in vitro of toxic macromolecules by strains of Beauveria bassiana, and purification of a chitosanase-like protein secreted by a melanizing isolate” Comparative Biochemistry and Physiology, Part C 138:149-161.
Fuguet, R., and Vey, A. (2004) “Comparative analysis of the production of insecticidal and melanizing macromolecules by strains of Beauveria spp.:in vivo studies” Journal of Invertebrate Pathology 85:152-167.
Gatehouse, A. M. R., Down, R. E., Powell, K. S., Sauvion, N., Rahbe, Y., Newell, C. A., Merryweather, A., Hamilton, W. D. O., and Gatehouse, J. A. (1996) “Transgenic potato plants with enhanced resistance to the peach-potato aphid Myzus persicae” Entomologia Experimentalis Et Applicata. 79:295-307.
Gietz, R. D., and Schiestl, R. H. (1994) “Transforming yeast with DNA” Methods in Molecular and Cellular Biology 5:255-269.
Grove, J., and Pople, M. (1980) “The insecticidal activity of beauvericin and the enniatin complex” Mycopathologia 70:103-105.
Gupta, S., Krasnoff, S., Underwood, N., Renwick, J. A. A., and Roberts, D. (1991) “Isolation of beauvericin as an insect toxin from Fusarium semitectum and Fusarium moniliforme var. subglutinans” Mycopathologia 115:185-189.
Hilder, V. A., Gatehouse, A. M. R., Sheerman, S. E., Barker, R. F. and Boulter, D. (1997) “A novel mechanism of insect resistance engineered into tobacco” Nature 330:160-163.
Hoffman, M. P. and Zalom, F. G. (1992) “Field evaluation of transgenic tobacco containing genes encoding Bacillus thuringensis delta-endotoxin or cowpea trypsin inhibitor-efficacy against Helicoverpa zea (Lepidoptera, Noctuidae)” J Econ. Entomol. 85:2516-2522.
James, C. (2003) ISAAA Briefs No. 30 (International Service for the Acquisition of Agribiotech Applications, Ithica, N.Y.).
Jaros-Su, J., Groden, E., and Zhang, J. (1999) “Effects of selected fungicides and the timing of fungicide application on Beauveria bassiana-induced mortality of the Colorado potato beetle (Coleoptera: Chrysomelidae)” Biological Control 15:259-269.
Kanaoka, M., Isogai, A., Murakoshi, S., Ichinoe, M., Suzuki, A., and Tamura, S. (1978) “Bassianolide, a new insecticidal cyclodepsipeptide from Beauvera bassiana and Verticillium lecanii.” Agric. Biol. Chem. 42:629-635.
Kucera, M., and Samsinakova, A. (1968) “Toxins of entomophagous fungus Beauveria bassiana” Journal of Inverebrate Pathology 12:316-320.
Leckie, B. M. (2002) “Effects of Beauveria bassiana mycelia and metabolites incorporated into synthetic diet and fed to larval Helicoverpa zea; and detection of endophytic Beauveria bassiana in tomato plants using PCR and ITS primers” Thesis, The University of Tennessee, Knoxville, Tenn.
Lewis, L. C., Berry, E. C., Obrycki, J. J., and Bing, L. A. (1996) “Aptness of insecticides (Bacillus thuringiensis and carbofuran) with endophytic Beauveria bassiana, in suppressing larval populations of the European corn borer” Agriculture, Ecosystem and Environment 57:27-34.
Lewis, L. C., and Bing, L. A. (1991) “Bacillus thuringiensis Berliner and Beauveria bassiana (Balsamo) Vuillimen for European corn borer control: program for immediate and season-long suppression” Canadian Entomologist 123:387-393.
Liu, D., Stephanie, B., Glancy, T., Li, Z., Hampton, R., Meade, T., and Merlo, D. J. (2003) “Insect resistance conferred by 283-kDa Photorhabdus luminescens protein TcdA in Arabidopsis thaliana” Nature Biotechnology 21:1222-1228.
McCoy, C. W., Samson, R. A., and Boucias, D. G. (1985) “Entomogenous fungi”, pp. 151-236, eds. Ignoffo, C. M., and N. B. Mandava, In Handbook of Natural Pesticides, Vol. 5. CRC Press, Boca Raton, Fla.
McDowell, J. M., Funderburk, J. E., Boucias, D. G., Gilreath, M. E. and Lynch, R. E. (1990) “Biological activity of Beauveria bassiana against Elasmopalpus lignosellus on leaf substrates and soil” Environmental Entomology 19:137-141.
Melander, M., Ahman, I., Kamnert, E., and Stromdahl, A. C. (2003) “Pea lectin expressed transgenically in oilseed rape reduces growth rate of pollen beetle larvae” Transgenic Research 12:555-567.
Pekrul, S., and Grula, E. A. (1979) “Mode of infection of the corn earworm by Beauveria bassiana as revealed by scanning electron microscopy” Invertebrate Pathology 34:238-247.
Perez, C. P. and Shelton, A. M. (1997) “Resistance of Plutella xylostella (Lepidoptera: Plutellidae) to Bacillus thuringiensis Berliner in central America” Journal of Economic Entomology 90:87-93.
Peferoen, M. (1997) “Insect control with transgenic plants expressing Bacillus thuringiensis crystal protein” pp 21-48, eds. Carozzi, N. and Koziel, M., In Advances in Insect Control, Taylor & Francis Ltd, London.
Poprawski, T. J., Greenberg, S. M., and Ciomperlik, M. A. (2000) “Effect of host plant on Beauveria bassiana- and Paecilomyces fumosoroseus-induced mortality of Trialeurodes vaporariorum” Environmental Entomology 29:1048-1053.
Quesada-Moraga, E., and Vey, A. (2004) “Bassiacridin, a protein toxic for locusts secreted by the entomopathogenic fungus Beauveria bassiana” Mycological Research 108:441-452.
Sane, V. A., Nath, P., Aminuddin and Sane, P. V. (1997) “Development of insect-resistant transgenic plants using plant genes: Expression of cowpea trypsin inhibitor in transgenic tobacco plants” Current Science 72:741-747.
Schroeder, H. E., Gollasch, S., Moore, A., Tabe, L. M., Craig, S., Hardie, D. C., Chrispeels, M. J., Spencer, D., and Higgins, T. J. V. (1995) “Bean alpha-amylase inhibitor confers resistance to the pea weevil (Bruchus pisorum) in transgenic peas (Pisum sativum)” Plant Physiology 107:1233-1239.
Stewart, C. N., Jr. (1999) “Insecticidal transgenes into nature: gene flow, ecological effects, relevancy and monitoring, pp. 179-190 In Symposium Proceedings No. 72 Gene Flow and Agriculture-Relevance for Transgenic Crops (Ed.: P. J. W. Lutman) Proceedings of a Symposium held at the University of Keele, 12-14 Apr. 1999. British Crop Protection Council, Surrey, UK.
Strasser, H., Vey, A., and Butt, T. (2000) “Are there any risks in using entomopathogenic fungi for pest control, with particular reference to the bioactive metabolites of Metarhizium, Tolypocladium and Beauveria species” Biocontrol Sci. Technol. 10:717-735.
Tabashnik, B. E. (1994) “Evolution of resistance to Bacillus Thuringiensis” Annual Review of Entomology 39:47-79.
Tabashnik, B. E., Liu, Y., Finson, N., Masson, L. and Heckle, D. G. (1997) “One gene in diamondback moth confers resistance to Bacillus thuringiensis toxins” Proc. Natl. Acad. Sci. USA 94:1640-1644.
Tabashnik, B. E., Malvar, T., Liu, Y., Finson, N., Bothakur, D., Shin, B., Park, S., Masson, L., Maagd, R. and Bosch, D. (1996) “Cross-resistance of the diamondback moth indicates altered interactions with domain II of the Bacillus thuringiensis toxins” Applied and Environmental Microbiology 62:2839-2844.
Vilcinskas, A. and Gotz, P. (1999) “Parasitic Fungi and their interactions with the insect immune system”, pp 267-313 In Advances in Parasitology Vol. 43. Academic Press, San Diego, Calif.
Xu, D., Xue, Q., McElroy, D., Mawal, Y., Hilder, V. A., and Wu, R. (1996) “Constitutive expression of cowpea trypsin inhibitor gene, CpTi, in transgenic rice plants confers resistance to two major rice insect pests” Molecular Breeding 2:167-173.
Zizka, J., and Wieser, J. (1993) “Effect of beauvericin, a toxic metabolite of Beauveria bassiana, on the ultrastructure of Culex pipiens autogenicus larvae” Cytobios 75:13-19.

We claim:

1. A composition of matter comprising a plant or plant part to which a composition comprising an extract of Beauvaria bassiana 11-98 (NRRL-30872) has been applied, said extract comprising insecticidal protein components having molecular weights of greater than 5000 daltons and said extract being insecticidal.

2. The composition of matter according to claim 1, wherein one or more additional pesticidal protein or pesticidal compound has been applied to said plant or plant part.

3. The composition of matter according to claim 2, wherein said one or more additional pesticidal protein or pesticidal compound is one or more insecticidal Bacillus thuringiensis (Bt) toxin or insecticidal portions thereof.

4. The composition of matter according to claim 1, wherein said composition further comprises an attractant and spores or crystals of Bacillus thuringiensis isolates.

5. A composition comprising an extract of Beauvaria bassiana, said extract comprising insecticidal protein components having molecular weights of greater than 5000 daltons and said extract being insecticidal when ingested by an insect.

6. The composition according to claim 5, wherein said composition further comprises one or more additional pesticidal proteins or compounds.

7. The composition according to claim 6, wherein said composition further comprises one or more insecticidal Bacillus thuringiensis (Bt) toxin or insecticidal portions thereof.

8. The composition according to claim 5, wherein said composition further comprises an attractant and spores or crystals of Bacillus thuringiensis isolates.

9. The composition according to claim 5, wherein said extract is an extract of Beauvaria bassiana 11-98 (NRRL-30872).

10. A method of treating a plant, plant cell, or plant part comprising the application of a composition comprising an extract of Beauvaria bassiana, said extract comprising insecticidal protein components having molecular weights of greater than 5000 daltons and said extract being insecticidal to a plant, plant cell, or plant part.

11. The method according to claim 10, wherein said composition further comprises one or more additional pesticidal proteins or compounds.

12. The method according to claim 10, wherein said composition further comprises one or more insecticidal Bacillus thuringiensis (Bt) toxin or insecticidal portions thereof.

13. The method according to claim 10, wherein said composition further comprises an attractant and spores or crystals of Bacillus thuringiensis isolates.

14. The method according to claim 10, wherein said extract is an extract of Beauvaria bassiana 11-98 (NRRL-30872).

15. A method of controlling an insect pest comprising the application of a composition comprising an extract of Beauvaria bassiana, said extract comprising insecticidal protein components having molecular weights of greater than 5000 daltons and said extract being insecticidal.

16. The method according to claim 15, wherein said composition further comprises one or more additional pesticidal proteins or compounds.

17. The method according to claim 15, wherein said composition further comprises one or more insecticidal Bacillus thuringiensis (Bt) toxin or insecticidal portions thereof.

18. The method according to claim 15, wherein said composition further comprises an attractant and spores or crystals of Bacillus thuringiensis isolates.

19. The method according to claim 15, wherein said extract is an extract of Beauvaria bassiana 11-98 (NRRL-30872).

20. The method according to claim 15, wherein said insect pest is coleopteran or lepidopteran.

21. A method of screening for an insecticidal protein comprising:
(a) transforming a host cell with one or more nucleic acid encoding B. bassiana proteins;
(b) culturing said host cell under conditions that cause the overexpression of said B. bassiana proteins;
(c) purifying B. bassiana proteins from said host cell or culture media;
(d) screening for at least one insecticidal protein comprising feeding an insect a diet comprising into which said at least one B. bassiana protein as been incorporated to form a protein modified insect diet and comparing the mortality of said protein modified insect diet on tested insects against a one or more of the following controls:
(i) a control diet into which no insecticidal proteins have been incorporated;
(ii) a control diet into which protein extracts from non-transformed host cells have been incorporated; or
(iii) a control diet into which protein extracts from host cells with a vector containing no nucleic acid inserts encoding B. bassiana proteins have been incorporated; wherein an insecticidal B. bassiana protein is identified when increased in mortality is observed in insects tested with said protein modified insect diet as compared to the mortality observed in insects fed one or more of the control diets.

22. The method according to claim 21, wherein said one or more controls is selected from: (i); (ii); (iii); (i) and (ii); (i) and (iii); (ii) and (iii); or (i), (ii) and (iii).

23. The method according to claim 21, wherein said host cell is a yeast cell.

24. The method according to claim 21, wherein said modified insect diet comprises a diet into which a pool of B. bassiana proteins is incorporated.

25. The method according to claim 24, wherein said pool of B. bassiana proteins comprises 100 to 200 recombinant proteins.

26. The method according to claim 21, wherein said insects are coleopteran or lepidopteran.

27. The method according to claim 21, wherein said insects are diamondback moths (Plutella xylostella), fall armyworms (Spodoptera frugiperda), corn earworms (Helicoverpa zea), southern corn rootworms (Diabrotica undecimpunctata), Colorado potato beetle (Leptinotarsa decemlineata), Asian tiger mosquitoes (Aedes albopictus), or green peach aphids (Myzus persicae).
